(19) United States
(12) Patent Application Publication
(10) Pub. No.: US 2023/0063822 A1
(43) Pub. Date: Mar. 2, 2023

(54) METHOD AND SYSTEM FOR TREATMENT OF USER'S EAR

(71) Applicant: Nicholas TIMMS, Encinitas, CA (US)

(72) Inventor: Nicholas TIMMS, Encinitas, CA (US)

(21) Appl. No.: 17/663,475

(22) Filed: May 16, 2022

Related U.S. Application Data
(63) Continuation of application No. 16/857,699, filed on Apr. 24, 2020, now abandoned.
(60) Provisional application No. 62/838,092, filed on Apr. 24, 2019.

(51) Int. Cl. A61F 7/02; A61F 7/10; A61F 7/08

(52) U.S. Cl. CPC A61F 7/02; A61F 7/10; A61F 7/08; A41D 20/00

(57) ABSTRACT

A securing device is shown and describe for positioning on a user's head. The securing device may be used to couple or attach an insert to the user's head in a desired position.

### CROSS-REFERENCE

This application is a continuation of application Ser. No. 16/857,699, filed Apr. 24, 2020, which claims the benefit of U.S. Provisional Application No. 62/838,092, filed Apr. 24, 2019, which are incorporated herein by reference in its entirety, and to which application priority under 35 USC § 120 is claimed.

### BACKGROUND

There are a number of sports activities that could result in trauma to a participant's ears. Activities that are generally most affected include grappling sports such as wrestling and jujitsu. Combat sports such as boxing or martial arts, as well as team sports such as rugby or basketball are also all susceptible to participants experiencing ear injury. In the case where a user receives trauma to their ear, it is common practice to apply endothermic therapy to the affected area. There may be other instances to apply heat or cold to a user's ears for medical treatment, such as an earache, or other ear condition.

Conventional systems for applying a temperature to a patient's body may include a material that encloses a heating or cooling substance. Typically, the substance is fully enclosed and integrated into the construction of the device for applying temperature. Therefore, once the temperature returns to atmospheric during use, then the entire device must be removed and reheated or cooled. Conventional systems may also not provide an easy way to clean the holder as the temperature gel may not be able to put through a washing machine. Conventional systems may also not provide a way to attach or secure the heating/cooling element in a desired location relative to the head and/or ear of the user. The heads of users may vary that securing a device around the head may not fit properly and slip during use. The temperature element may therefore not be securely positioned relative to a desired position on the user's head.

### SUMMARY

Exemplary embodiments include a securing device with pockets aligned with and/or over the user's ears. These pockets would allow the user to comfortably add and wear a hot or cold pack over one or both ears to aid in applying a temperature directly on and/or around the ear region.

Exemplary embodiments herein include a securing device that can be used to aid in the therapeutic recovery from trauma suffered directly to the user's ears. In cases like this, it may be desirable to provide a medical headband that specifically targeted the user's ears and whose design would allow the user to quickly replace the exhausted hot or cold packs with productive fresh ones. This would provide instant and continuous relief to the user.

In accordance with an embodiment, the securing device in a form of a headband is disclosed. The headband includes a main body with two distinct pockets over the wearer's ears which are designed to securely compress removable packs within a pocket against the wearer's ears.

The main body of the headband may be designed to be worn comfortably on the head of the user. The body may have two pockets coupled thereto and positioned on each side of the wearer's head—appropriately over the user's ears. The body may be configured to secure the device to a wearer's head, comfortably and with reduced slippage.

The individual hot packs or cold packs are configured to be received inside each pocket to provide relief to the user.

In some embodiments, the body can be made from a material that is stretchable. The two pockets can be defined using an endothermic conducive material and using side stitching on the inside of the headband that would create openings that would allow for a hot/cold pack to fit inside. Each pocket can also include a fastener to help close the pocket to help retain the movement of the pack if the user desires.

In some embodiments or aspects, the temperature element heating or cooling pack can include a shell and fill material, wherein the fill material is enabled to provide either a warming or cooling sensation to the user. The fill material can be gel, gel-filled beads, fluid, water, or a combination thereof which can be cooled or warmed depending on the user's desired application. Additionally, the fill material can include components capable of warming or cooling via endothermic reaction.

These and other purposes, goals and advantages of the present application will be apparent from the following detailed description of example embodiments read in connection with the accompanying drawings.

### DRAWINGS

FIG. 1. illustrates a perspective view of an example securing device without any temperature packs present.

FIG. 2. illustrates a perspective view of an example securing device with temperature packs partially inserted and outside of the securing device.

FIGS. 3A-3B illustrate front and rear perspective views, respectfully, of a securing device in a worn position on a wearer's head.

FIG. 4 illustrates a cross-sectional view of the exemplary securing device from FIG. 3B along line A-A with a temperature pack inside the pocket against the user's ear.

FIGS. 5A-5B illustrate exemplary cross sections of an exemplary securing device with a temperature pack partially and fully positioned therein from an inside view of the securing device that would be against a wearer's head in an in use position.

FIGS. 6A-6B illustrate exemplary front views of a securing device in a flat position to illustrate different configurations of the securing device.

FIGS. 7A-7C illustrate exemplary side views of a securing device in a flat position to illustrate different configurations of the securing device.

FIGS. 8A-8C illustrate exemplary rear views of a securing device in a flat position to illustrate different configurations of the securing device.

FIG. 9 illustrates an exemplary pattern for creating a securing device according to embodiments described herein to illustrate exemplary relationships between relative sides and edge portions.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention, and are not limiting of the present invention nor are they necessarily drawn to scale.

Exemplary embodiments include a securing device designed to help athletes with recovery from post-athletic activity. A securing device is shown and described in the form of a headband having pockets designed to be worn over the wearer's ears to allow a temperature element to be inserted into the designated pockets. The system including the securing device and temperature elements may be configured to allow the user to seek relief from trauma caused on, or around the user's ears. Although exemplary embodiments described herein are configured for medical applications including trauma relief, the invention is not so limited. For example, other use cases to shield the ear or apply heat, cold, or pressure to the ear are also within the scope of the instant invention. For example, the pockets provided in the instant securing device may also be used to insert insulation for reducing cold to contact the ears in winter environments, or to insert insulation to reduce noise penetration to the ear drums. Other applications for heat or cold application may also be used herein.

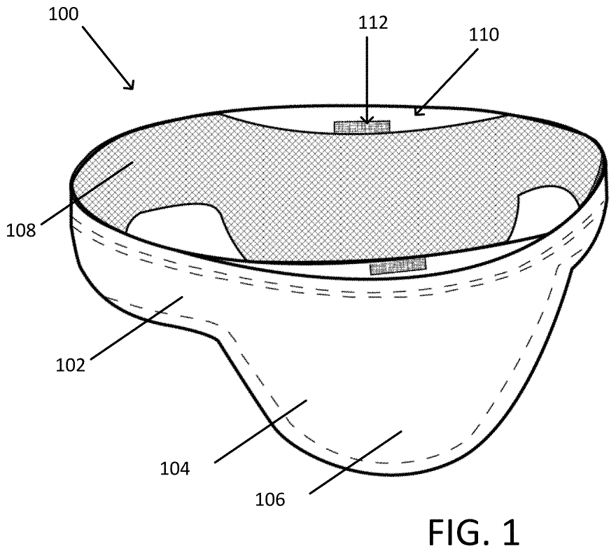

FIG. 1 illustrates a perspective view of an example securing device 100 without the temperature element present. The securing device 100 is designed to provide the user a combination of functions including comfort and relief. The securing device 100 may include a body 102 for positioning around a wearer's head, and projections 104 configured to be positioned over a wearer's ears. The exemplary securing device may comprise materials that are stretchable to help to conform to the shape of the user's head. This material can be made from synthetic and/or manmade materials, natural materials, and/or blended combinations such as, for example, acrylic, nylon, spandex, cotton, other natural or synthetic materials. The material may be chosen for comfort, flexibility, construction, temperature insulation, temperature conduction, and combinations thereof. In some embodiments, the securing device is made of nylon and spandex.

The body 102 can be constructed from one piece of material or multiple layers of material. In an exemplary embodiment, the body 102 is constructed of an outer layer 106 and an inner layer 108. In an exemplary embodiment, the outer layer and inner layer are coextensive to reduce seams and/or terminal ends of material to reduce potential discomfort as the body is positioned against a wearer's head. In an exemplary embodiment, the inner layer is positioned over the projection 104 only and/or at the projection and a portion of the body adjacent to the projection to create the pocket for the temperature element. The inner layer may not cover a front and/or back portion of the circumferences of the securing device.

In an exemplary embodiment, the outer layer 106 and inner layer 108 are configured to form a pocket 110. The pocket includes an opening at and/or adjacent a top edge of the securing device 100 and a closed end at or adjacent a bottom edge of the securing device 100. The top and bottom are up and down relative to a user in a worn position sitting up, such that the top is toward the top of the head of a wearer, and the bottom is toward the lower body of a wearer in the worn position.

In an exemplary embodiment, the inner layer 108 may be configured to propagate a temperature change more easily than the outer layer 106. For example, the inner layer 108 may be of a more thermally conductive material than the outer layer 106. The inner layer may also include holes, apertures, or otherwise be more porous or less dense than the outer layer. The inner layer may include a mesh, or other configuration of holes or other apertures to transmit a temperature to the interior of the securing device. The inner layer and outer layer may also be of the same materials and/or configurations.

The securing device 100 body 102 has two pockets 110 that may be similar in size and configured to hold removeable temperature element in a predetermined configuration that may position the temperature element over the user's ears. The temperature elements may be hot and/or cold packs that may be inserted into the pocket 110 opening. In an exemplary embodiment, the pocket 110 may include a securing element 112. The securing element 112 may be positioned at or adjacent the opening of the pocket near or at the top of the securing device 100. The securing element made be, for example, hook and loop fastener, Velcro®, button, snap, magnet, or other fastening device that would allow the user to securing the pocket opening in a closed configuration and release to open the opening, permitting the separation of the inner layer from the outer layer and provide access to the interior cavity of the pocket. It is not necessary for the securing element 112 to be activated for the securing device 100 to be functional, but it may help to keep the temperature element within the pocket, and/our secure the temperature element is a desired location within the securing device to define a specific position relative to the body 102 and/or the wearer's head and/or the wearer's ear.

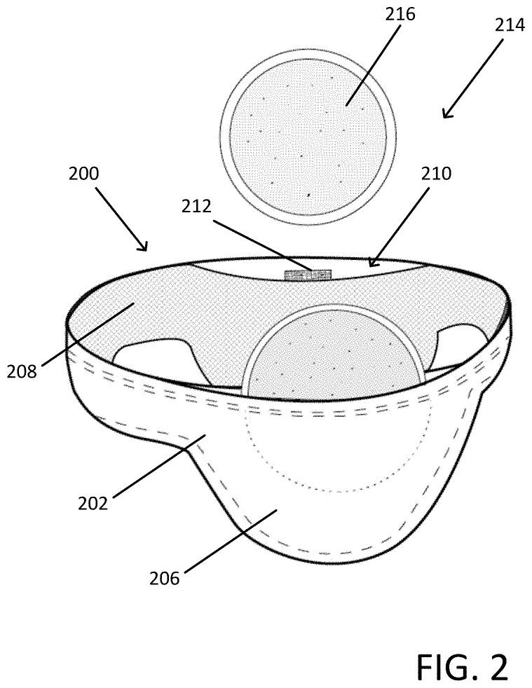

FIG. 2 illustrates a treatment system 214 according to embodiments described herein including a securing device 200 and temperature element 216. As illustrated, the system include two temperature elements, one configured to be positioned in each pocket 210. As illustrated, one temperature element is positioned outside of the securing device, and another is positioned partially in the pocket, extending out of the opening. When an exemplary temperature element 216 is fully inserted into a pocket 210, the body 202 may fully encompass or enclose the temperature element. In an exemplary embodiment, when fully inserted, the temperature element may be positioned at or near the bottom of the pocket against a lower edge of the securing device. The securing element may also be used, as described herein, to position the temperature element away from the bottom edge of the pocket in a specific or desired location within the pocket relative to the securing element 212.

The pockets 104 of headband 100 may have an approximate opening of 6¾″. Each pocket may include one, two, or more stitches. As illustrated, the top of the securing device 200 includes two stitches, one to secure each of the layers in a fold-over position, and one or two additional stitches to secure the layers together. The lower edge of the securing device may also include one, two, or additional stiches to secure the layers together. The terminal edges of the securing device, may also include fold over seams. The fold over seams may provide a soft, elastic, or a combination thereof, to permit the securing device to comfortably conform to the wearer's head in an in use position.

In an exemplary embodiment, an exemplary securing device 200 includes an outer layer 206 and an outer layer 208. The outer layer 206 may be sewn together with a similarly sized of material defining the inner layer 208. The inner layer 208 may be a piece of endothermic friendly material. The dual layer securing device may be folded in half to form the exterior and the interior layers and sides of the securing device configured to circumscribe the head of a wearer in an in use position. The top of the pocket 210 can be formed by folded hems along with reinforced double stitching and/or single stitching.

The construction of the securing device may include double stitching to secure the folded hem that fastens the inner layer or endothermic friendly material on the inside to form pockets designed specifically to fit the temperature elements. The endothermic friendly material may be constructed from a material that would be conducive or facilitating the thermic transmission from the temperature element to the wearer's head. The endothermic friendly material may also be stretchable and flexible to provide stability and comfort to the user while the securing device 200 is being worn. Additionally, the securing device may include stitching to reinforce a hem that runs along the bottom of the headband to help support the bottom of the pocket of the securing device.

In an exemplary embodiment, the temperature element 216 includes a heating or cooling pack. The heating or cooling pack may include a shell and fill material, wherein the fill material is configured to provide either a warming or cooling sensation to the user. The fill material can be gel, gel-filled beads, fluid, water, or a combination thereof that can be cooled or warmed depending on the user's desired application. Additionally, the fill material can include components capable of warming or cooling via endothermic reaction.

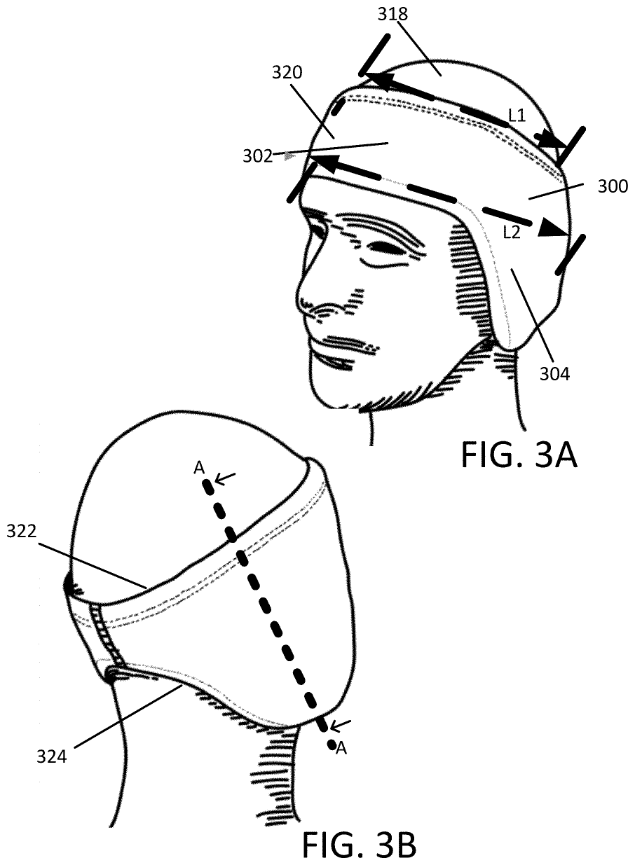

FIGS. 3A-3B illustrates an exemplary securing device 300 positioned on a wearer's head 318. As illustrated in FIGS. 3A-3B, the material and the shape of the securing device 300 is configured to conform to the user's head 318. The securing device 300 includes a body 302 that is configured to circumscribe a wearer's head 308. The securing device includes projections 304 that are defined by a wider section of the body that are configured to be positioned over a wearer's ears (not seen as covered by the projections). The body 302 and projections 304 are configured to be positioned over the wearer's ear such that temperature elements positioned within an interior cavity of the projection may be positioned over a wearer's ear and apply a temperature to the wearer's ear. The system may therefore be configured to deliver a beneficial application of a temperature change from the inserted hot/cold packs, as described herein.

In an exemplary embodiment, as seen in FIG. 3A, a front portion 320 of the body 302 may include a curved edge, such as along the upper edge 322 of the securing device or a lower edge 324 of the securing device. The curved shape of the front of the headband may be configured to conform to the wearer's forehead more comfortably (as compared to a shape in which the upper edge and/or lower edge were simply a horizontal line from temple to temple). Exemplary curvatures are described herein. The exemplary curvatures may be configured to permit the circumference of the device aligned with a portion of the upper edge 322 to be less than a circumference of the device along and/or aligned with a portion of the lower edge 324. The respective circumference may be along a portion of the upper and lower edges of the body 302 outside of the projections 304, such as along the edge over the forehead or center of the wearer's head above the eyes, when in an in use configuration. As seen in FIG. 3A an exemplary circumference is partially represented by dotted lines L1 and L2. Therefore, as illustrated L1 is less than L2 so that the securing device is configured to be positioned on the wearer's head. The securing device may be configured to interact and engage the natural configuration of the wearer's head to provide support to the device. For example, the smaller circumference may interact with the narrower portion of a wearer's head and reduce the downward slippage of the device when positioned on the wearer's head as the smaller circumference at the upper edge relative to the lower edge may reduce or prevent the device from moving further down on a wearer's head during use. As described herein, the different circumference may be achieved in different ways.

When the user fully inserts both temperature elements correctly into each interior cavity of the projection 304, then each inserted temperature element can be directly over the user's ears. Depending on if the inserted temperature element is a hot or cold pack, there can be an endothermic reaction emitted through the endothermic friendly material of the inner layer of the projection that can cause the temperature of the user's ears to rise or fall. The temperature of hot or cold pack may depend on the desired outcome of the user. A user may want their ears to be warmed if the ambient temperature is too cold or to induce blood circulation. Conversely, the user may want to cool their ears to help to promote a reduction in inflammation due to trauma caused to the ear region.

FIG. 3B illustrates a view from the back of the user's head 318. In an exemplary embodiment, the body 302 is made of two layers of co-extensive material that wrap around the users head to create a single continuous piece of material that can be coupled directly together. FIG. 3B illustrates an exemplary back of the securing device 300 with a reinforced hem along the terminal ends of the layers to directly couple the ends together. The width of the body at the back hem may be a minimum width compared to the rest of the body. The width of the body at the back portion, such as at the hem may be less than a width of the body at the projection area, and/or the width of the body at the front portion 320 of the body 302.

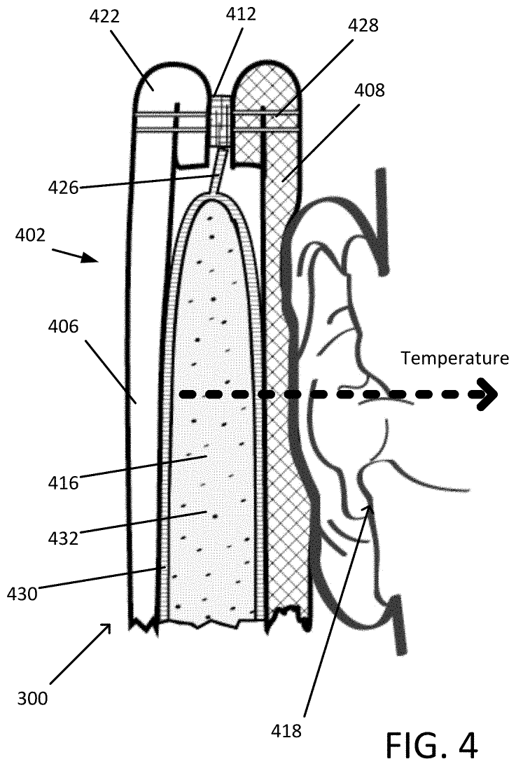

FIG. 4. Illustrates an enlarged, not to scale cross-sectional view along line A-A of FIG. 3B of a securing device 300 with a temperature element 416 positioned within an interior cavity of the body. The interior cavity may be defined between an inner layer 408 and an outer layer 406. The system may be configured to position he temperature element 416 over a user's ear 418 to apply a temperature of the temperature element to the user.

The inner layer 408 and outer layer 406 may be coupled with a securing element 412 to permit the interior cavity to be opened and closed. In an exemplary embodiment, the temperature element may include a positioning element 416. The positioning element 426 may be configured to engage with the securing device 300 to position the temperature element 416 is a desired position relative to the securing device. For example, the positioning element 426 may interact and/or engage with the securing element 412. The positioning element may include sides that couple between the securing element such that it becomes an intermediary between the sides of the securing element. The positioning element nay also be configured to interact and/or engage with the securing element by being positioned in a desired position relative to the securing element. For example, the positioning element, may be an aperture or a plurality of apertures to permit the securing element to pass through or partially through the positioning element. In an exemplary embodiment, the positioning element is a perimeter extension about a circumference of the temperature element. The perimeter extension may be of a material on one or both sides to engage and interact with Velcro® of the securing element. Other embodiments of a positioning element 426 may also be used. For example, one or both sides of the exterior surface of the temperature element may be textured to frictionally engage within one or both interior sides of the inner surface of the cavity formed by the inner and outer layers. The temperature element may include Velcro®, hook and loop fastener, textured surface, adhesive surface, snap, button, or other engagement mechanism to retain the temperature element in a desired position within the cavity of the projection.

As illustrated, the securing device 300 may include a hem 428 that circumscribes all or a portion of the top portion of the securing device. The hem may be at a fold over portion of the inner and/or outer layers of the securing device and/or may be a stitching about a top portion 422 of the securing device. The hem may directly couple a portion of the inner layer and the outer layer about a portion of the circumference of the securing device, and may indirectly couple another portion of the inner layer and the outer layer about another portion of the circumference of the securing device. The other portion of the hem that indirectly couples the inner layer and the outer layer may be configured to create the opening that permits access to the interior cavity formed between the inner and outer layers. The securing element may be portioned within this other portion of the of the circumference. When combined together, the user can secure each side of the pocket together to secure the inserted temperature element.

In an exemplary embodiment, the hem 428 about an upper edge 422 of the securing device may be configured to reduce the stretchability of the body 402. As shown, the securing device 300 may include a body 402. The body 402 may be of one or more layers. The one or more layers may be of a stretchable material to permit the securing device to conform and fit various user's head. The layers may be sewn together. In an exemplary embodiment, the hem 428 at an upper edge of the securing device may be configured to permit a limited stretch. A limited stretch may permit the material to realize some of its stretch but may create a reduced stretch relative to the material without the hem. The reduced stretch may be created through the sewing, or in the fold over of the material itself, and combinations thereof.

Exemplary embodiments of the temperature elements 416 include hot and/or cold packs. The temperature element 416 may include a shell 430 and a fill material 432. The shell 430 may be a flexible material configured to retain and enclose the fill material 432. The fill material may be a reusable material that can be repeatedly heated or cooled, or may be a single use material, such as an exothermal chemical reaction. In the reusable case, the fill material can be cooled with refrigeration (or by freezing) or warmed by a microwave or another type of heating source. The fill material 432 can include a gel, gel-filled beads, fluid, water, or another type of fill material that would have hot or cold properties. The shell 430 is generally flexible to allow it to better conform to the user's ears.

In an exemplary embodiment, the temperature element 416 may be a generally round or ovoid shape. In an exemplary embodiment, the temperature element has a diameter of 4 inches. However, the temperature element 416 can be of any shape or size including rectangles, polygons, ellipticals, squares, or any combination of shapes that could fit into the pockets or even exceed the pocket size. In an exemplary embodiment, the temperature element 416 comprises a gel pack that can be cooled/frozen and/or warmed depending on the intended use. Hot/cold packs are generally flexible to allow it to conform to the ears of the user once it has been inserted into a pocket of the securing device. Once the temperature element 416 has been inserted between the inner layer 408 and outer layer 406 of the securing device 300, the temperature can propagate a temperature difference through the inner layer 408 toward the user's ear as indicated by the dashed line arrow of FIG. 4.

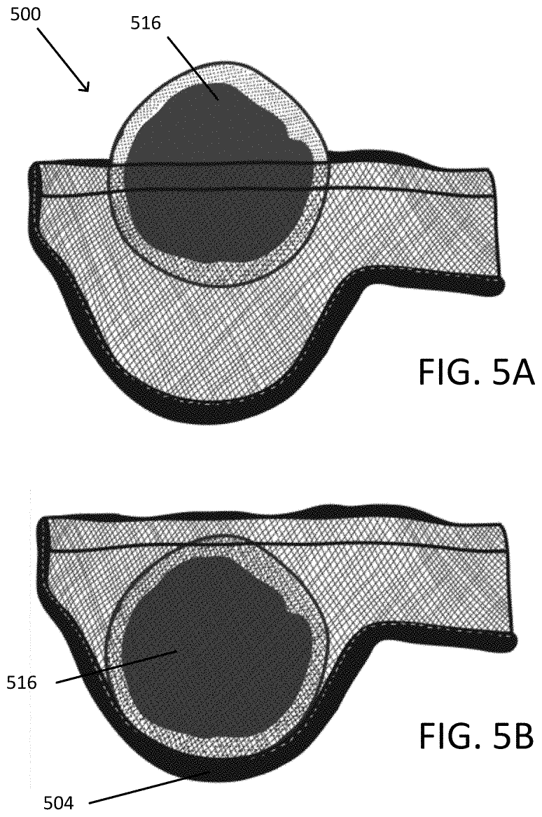

FIGS. 5A-5B illustrate a partial view of an exemplary securing device 500 with a temperature element 516 partial and fully positioned in a cavity of the securing device. As illustrated, the temperature element 516 through an opening to provide access to the interior cavity of the securing device created between an inner and outer layer. When fully positioned within the securing device, the temperature element may rest against a bottom of the securing device. The temperature element may be sized such that when the temperature element is positioned contacting a bottom edge of the securing device, a central region of the temperature element is positioned over a portion of the user's ear in an in use position. As seen in FIG. 5B, the temperature element 516 may be approximately the same size as the body at the projection 504 of the securing device 500.

Exemplary embodiments of the securing device have unique cuts to create a more comfortable and/or secure fit of the securing device to the wearer's head. In an exemplary embodiment, the cut may be configured such that a circumference along a upper edge is smaller than a circumference at a bottom edge. The securing device may create a generally band or strip shaped portion with two projections extending therefrom. The upper and lower edges for comparison of the circumference may be along or approximate the upper and lower edges of the band portions, where the lower edge may extend through a portion of the projection. In an exemplary embodiment, the top circumference is approximately 0.5-1.5 inches smaller than the lower circumference. In an exemplary embodiment, the top circumference is approximately 18 inches while a lower circumference is approximately 19 inches. Having a difference between the top circumference and the bottom circumference causes a taper effect may improve the securing device's comfort and function to keep the temperature elements close to the user's ears. The exemplary sizes provided herein are for an adult average size head. Other sizes may be used depending on gender, age, etc.

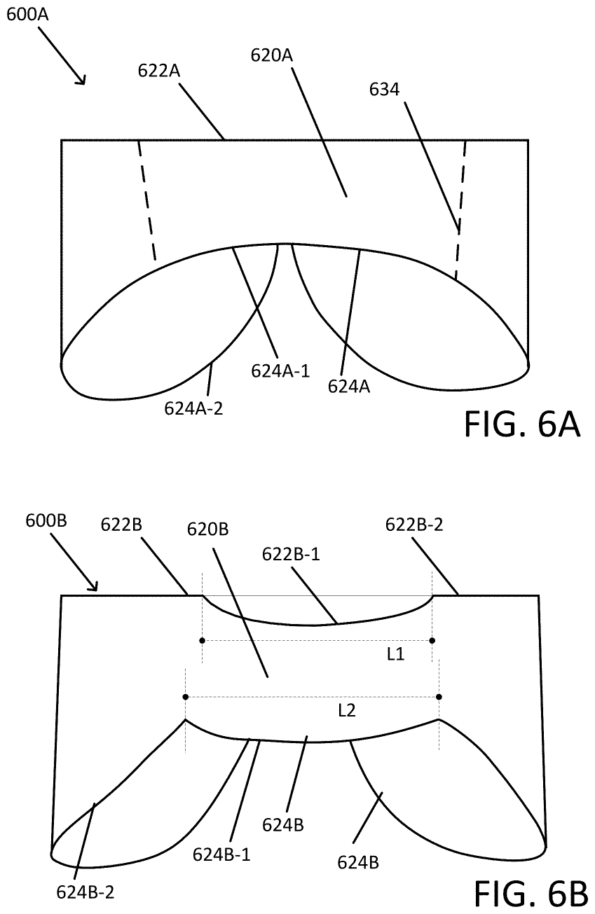

FIGS. 6A-6B illustrate exemplary front profiles when the securing device 600A, 600B is laid flat. The exemplary embodiments are configured to conform to a user's head and provide a more secure and/or comfortable securing device. FIGS. 6A-6B illustrate exemplary curved front portion 620A, 620B that creates the different circumferences along the upper edge 622A, 622B and lower edge 624A, 624B.

FIG. 6A illustrates a first configuration with a straight cut upper edge 622A and a curved lower edge 624A. The curved lower edge may include a first portion 624A-1 along the front portion 620A of the securing device 600A, and two second portions 624A-2 on opposite sides of the first portion 624A-1. The two second portions 624A-2 define the projections configured to extend downwardly away from the upper edge 622A and cover the user's ears. The second portions 624A-2 may curve downward and then turn back up at the back portion of the securing device. The lower edge 624A may therefore device a concave downward curve along a front portion 620A of the device and two convex downward curved portions on opposite sides of the securing device. Downward is being used as a reference frame for the direction of curvature if looking at the device from below toward the lower edge, such that a convex downward curved edge is generally a u shaped curve. A curvature upward would be a reference frame for the direction of curvature is looking at the device from above toward the upper edge and would create an opposite concavity from the downward reference frame. As illustrated, the securing device may also include hem 634 to separate the pocket from the rest of the securing device and retain the inserted temperature element in a desired location.

FIG. 6B illustrates a second configuration with the front portion having curved upper and lower edges. As illustrated, the securing device 600B has an upper edge 622B that includes a tapered second portion 622B-2 that extends around a majority of the circumference of the device. The front portion 620B may correspond to a first portion 622B-1 of the upper edge 622B. The first portion 622B-1 may be curved and may dip downward toward the lower edge 624B. The first portion 622B-1 may be concave upward. The lower edge 624B may similarly have a concave upward curve along a first portion 624B-1 of the lower edge along the front portion 620B of the securing device 600B. The portion along the respective edges along the front portion 620B may be configured to create the different circumferences between the upper and lower edges. For example, length along the curve of the first portion 622B-1 along an upper edge may be less than the length along the curve of the first portion 624B-1 along the lower edge. The difference in length may be achieved by making the transverse, linear length of the first portion 622B-1 along the upper edge 622B (L1) less than a transverse, linear length of the second portion 624B-1 along the lower edge 624B (L2). The difference in circumference between the upper and lower edges may be achieved by making the radius of curvature of the first portion 622B-1 along the upper edge 622B less than the radius of curvature of the first portion 624B-1 along the lower edge 624B. The difference in circumference between the upper and lower edges may be from a combination of transfer, linear length of the respective curved portion and/or in the radius of the respective curved portions. The lower edge may similarly have two second portions 624B-2 on opposing sides of the first portion to define the projections. The two second portions 624B-2 may be concave downward. The lower edge may also include a third portion, not seen, that is concave downward between the two second portions 624B-2.

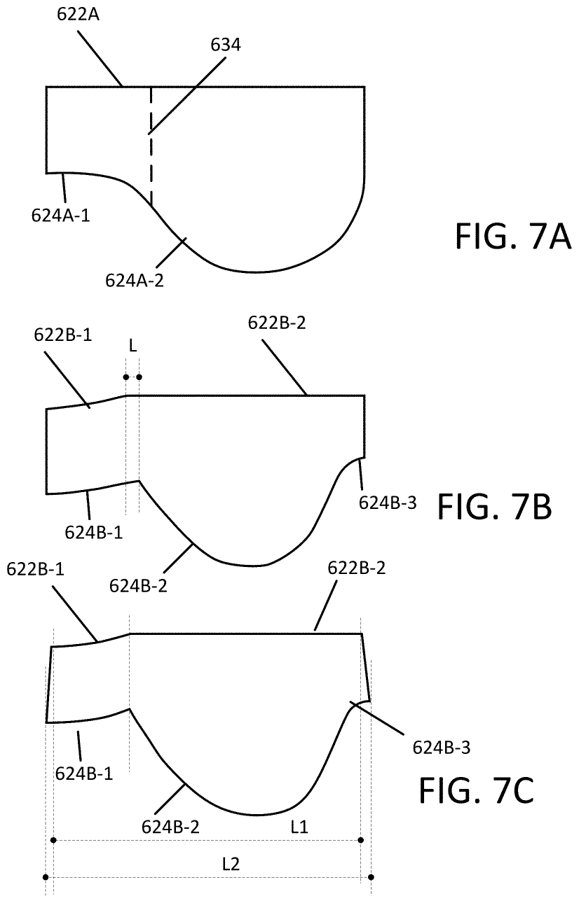

FIGS. 7A-7C illustrate exemplary side profiles when the securing device is laid flat. The exemplary embodiments are configured to conform to a user's head and provide a more secure and/or comfortable securing device. FIGS. 7A-7C illustrate exemplary curved front portion and back portions that creates the different circumferences along the upper edge 622A, 622B and lower edge 624A, 624B.

FIGS. 7B-7C illustrate exemplary upper and lower edges having a curved front portion. As illustrated in FIG. 7B the first portion 622B-1 have ends along an upper edge 622B that occur within the ends of the first portion 624B-1 along the lower edge 624B; while the ends of the first portion 622B-1 of the upper edge 622B are coextensive with the ends of the first portion 624B-1 of the lower edge 624B as illustrated in FIG. 7C. FIGS. 7B and 7C illustrate a third portion 62B-3 along the lower edge 624B that is concave downward along the back of the securing device on a side opposite the front portion 620B. FIG. 7C also illustrate an additional taper imposed between the top of the device and the bottom of the device, resulting in the linear, transverse length along the top of the body L1 is less than the linear, transverse length along the bottom portion of the body L2.

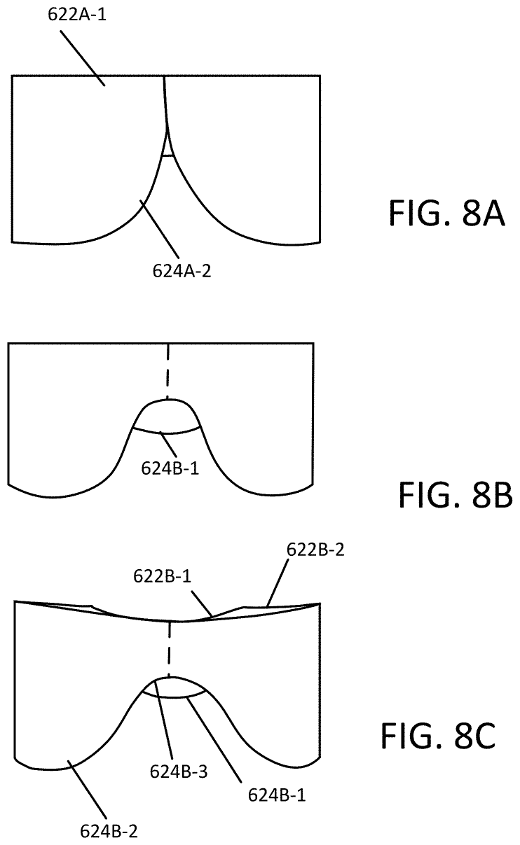

FIGS. 8A-8C illustrate exemplary rear profiles when the securing device is laid flat. FIG. 8A illustrates a linear upper edge 622A-1 and a lower edge having a two second portions 624A-2 that directly meet in a rear of the securing device. FIGS. 8B-8C illustrate a lower edge having a third portion 624B-3 that is concave downward positioned between the two convex downward portions that define the projections. FIG. 8C illustrates the configuration of the upper edge that has both a curved first portion 622B-1 along a front portion and a tapered second portion 622B-2.

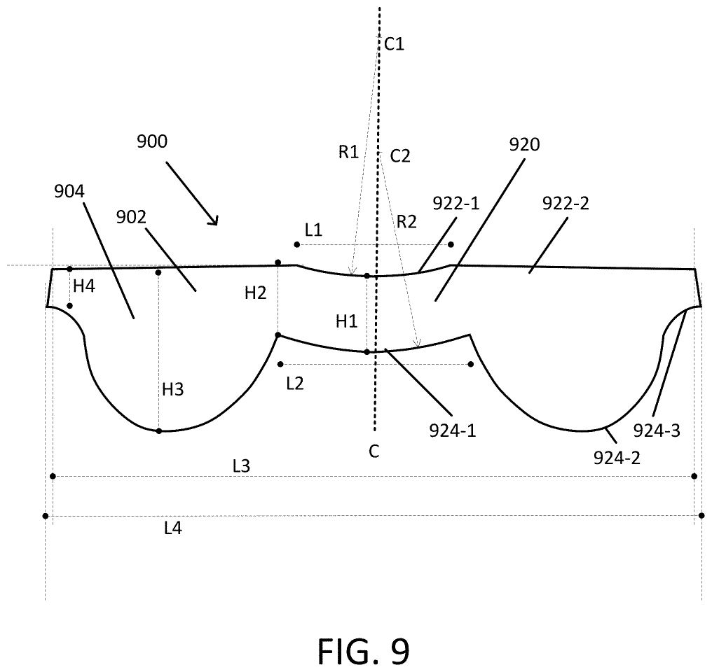

FIG. 9 illustrates an exemplary pattern for creating a securing device according to embodiments described herein. As illustrated, the securing device 900 may include a body 902 having a front portion 920 and projection sections 904. The front portion 920 is configured to position on a user's forehead and the projection sections 904 are configured to be positioned over a user's ears in an in use configuration. The front portion 920 includes an upper edge 922-1 and a lower edge 924-1, where the upper edge 922-1 and lower edge 924-1 are curved, concave upward. The transverse, linear length (L1) of the front portion 922-1 upper edge is less than or equal to the transverse, linear length (L2) of the front portion 924-1 lower edge. The front portion upper edge 922-1 and front portion lower edge 924-1 may each have a radius of curvature. The radius of curvature R1 of the front portion upper edge 922-1 may be less than or equal to the radius of curvature R2 of the front portion lower edge 924-1. The center of curvature of the respective front portions may be concentric or may be different. Any combination of the position of the center of curvature, the transverse, linear length, and/or the radius of curvature of the front portions of the lower and upper edges (924-1 and 922-1) are configured such that a circumference about an upper edge is less than a circumference about a lower edge. FIG. 9 also illustrates the projections 904 defined by a second portion 924-2 of the lower edge. The back portion of the securing device is defined by the direct contact of terminal ends of the second portion defining the projections (as seen in FIGS. 6A, 7A, and 8A) or by a third portion positioned between the projections and defined by a concave downward portion of the lower edge 924-3. When configured, the front portion may have a height H1 in a middle of the front portion that is equal or greater than a height H2 at opposing ends of the front portion. The height at a back portion H4 may be a minimum, while the height H3 at the projections may be a maximum. The top edge 922-2 outside of the front portion may be generally straight and may either be generally horizontal or slightly tapered. The terminal ends of the securing device that directly attach at a back of the securing device may also be straight downward or may slightly flare or extend outward along a lower edge. The transverse, linear length along an entire top edge of the securing device (L3) may be less than or equal to the transverse, linear length along an entire lower edge of the securing device (L4).

In an exemplary embodiment, the length along the upper edge is less than the length along a lower edge. The upper edge length L3 may be approximately 18 inches, while a lower edge length L4 may be approximately 19 inches. Different sizes may be used depending on the target user group, based on age, and/or gender or normal, small, or large sizes. The upper edge length L3 may be less than the lower edge length L4 by 0.25 inches to 1.5 inches, or approximately an inch.

The front of the head band may include a subtle contour, in the form of a curvature along the upper edge, the lower edge, or both the upper and lower edge. The purpose of this curvature is to help improve comfortability of the head band to the wearer's head while being worn (versus a horizontal straight cut that will force the head band to sit too closely to the top of the crown of the user's head). The front of the head band is noticeably thinner in height (approximately 2.25 inches) in comparison to the pockets that cover the ear's of the user. The approximate width of the front of the head band may be approximately 5.5 inches while the linear length of the top of the curved front of the head band is approximately 5.25 inches and the bottom of the curved front is approximately 5.5 inches. The height of the strip of material that directly touches the user's forehead is approximately 2.25 inches.

Moving from the front of the head towards the ears. On each side of the securing device, there may be two pockets made from a similar flexible outer material as the main body. These pockets completely cover the user's ears. The pockets begin at each end of the curved forehead strip. There is a noticeable height difference between the height of the front of the securing device and where the pockets over the ears lie. In some cases, the liner height of the pockets at the projection 904 (H3) are approximately 5 inches (between 3-6 inches) compared to the height of the front of the securing device H1 of approximately 2.25 inches (between 1.5 to 3 inches).

The pockets are generally round or elliptical in shape having an approximate diameter of 5 inches (3-6 inches) and a height H3 of 5 inches (between 3-6 inches). The securing device may taper after the projections 904 toward the back of the securing device. The height at the back of the securing device may be approximately 1.75 inches (between 1-3 inches).

The inside of the securing device, that is, the material that directly touches the user's head, is generally made of a flexible, yet thin material. The material is selected for its endothermic friendly characteristics which will help to maximize the cooling or warming properties of the hot/cold packs to pass through the material directly to the user's ears. This material can be flexible and breathable. Examples of the inner material include mesh, nylon, or spandex. The inner material may include holes (such as from cut apertures or a porous material) to assist in the temperature transfer from the temperature element to the user's head.

The pockets that hold the hot/cold packs have openings that are approximately 6.74 inches (between 3-7 inches). The vertical height of the projections 904 are approximately 5 inches while the linear horizontal length is approximately 5 inches.

The securing device may include a hem reinforced by single or double stitching that runs along the upper edge and the lower edge to help define the shape of the projection. This helps to secure the hot/cold pack once it is fully inserted into the cavity of the projection—that is, when the hot/cold pack is touching, or close to touching the bottom hem of each projection. In some cases, there is a securing element that utilizes Velcro®, hook and loop fastener, a button, a snap, a magnet or other mechanical device that help to secure the top of each side or layer of the projection together, thus forming a closable pocket. This securing element may be used to secure the temperature element inside the pocket which is helpful to the user when the securing device is taken on or taken off. The interior of the pocket and/or surface of the inserted hot or cold pad may also be textured or have an attachment mechanism for retaining the inserted hot or cold pad in a desired location relative to the pocket.

There are a number of applicable uses for the securing device according to embodiments described herein. For example, the securing device may be used as a medical head band that allows the user to reap the benefits of compressing either a hot or cold pack against their ears.

One of the biggest voids in the marketplace is an accessible solution that would allow a user to comfortably and productively compress a cool pack to an injured ear suffered from blunt force trauma. This is especially common for athletes who participate in combat or grappling sports such as wrestling, Brazilian Jujitsu, Judo, Sambo, boxing, muay thai, kickboxing, mixed martial arts (MMA) just to name a few where direct strikes or abrasive movements are well known to constantly crush and damage the fragile cartilage within the participating athlete's ear. Additionally, similar trauma to an ear during athletic activity is not only limited to combat or grappling sports, but similar injuries can also be found in team sports. Participants in a (non-helmeted) team sports such as rugby or basketball are also vulnerable to strikes to the head—and subsequently, the ear of the participating athletes.

In all cases, the most recommended methods include immediately treating the injured area to reduce the inflammation to one's ears (or its immediate region) by directly cooling the area using cold therapy. Cold therapy helps to constrict the blood vessels, reduce swelling and decrease inflammation. It can also numb the area to reduce pain. Using an ice or a cold pack is also beneficial for managing flare-ups of chronic conditions and thus reduce the beginning stages of an aural hematoma (also known as “cauliflower ear”) to the athlete's ear.

Conventionally, in order to apply cold therapy to one's ear, the person would typically need to remain immobile while holding a cool pack directly to their ear for 15-20 minutes. Not only is this stationary action uncomfortable, it also severely impairs one's ability to multi-task during treatment. Because the injured person uses one arm to hold the compress against their ear, they are limited to only one free arm. Using exemplary embodiments of a securing device described herein as a method to apply cold therapy to treat inflammation directly to the ear region allows the user to be able to utilize both their arms freely, as well as allow the user to move around while simultaneously getting cold therapy treatment for their injury.

Similar to cold therapy, compressing a warming pack to one's ear also holds a number of beneficial values to the user. One example can be found when the user wears the head band outdoors in snowy or frigid conditions—especially during times where they are exposed to wind chill (such as the idle time spent on a ski lift). Additionally, compressing a warm pack to a child's ear who is suffering from an earache has also proved itself as a valuable application for exemplary embodiments described herein. The immediate relief that the warmth brings to the child is therapeutic and calming and allows the child to relax from the pain suffered from a common earache. In addition to helping to relax the child, the design of the exemplary securing device allows the child to be comfortable, to be mobile, to have the ability to use both arms freely, and also permits the child to sleep safely while wearing the securing device, should they decide to do so.

An exemplary embodiment includes a securing device configured to be positioned on a wearer's head, including a body configured to circumscribe the wearer's head, and two projections on opposing sides of the body, the two projections each defining a cavity, the projections configured to be positioned over a wearer's ears when the body is positioned to circumscribe the wearer's head, each of the two projections configured to receive an insert.

The insert may be a temperature element. The body may include a material layer and the cavity of each of the two projections may be created by an inner layer. The inner layer may include holes to facilitate thermal transfer from the temperature element to the user's head. The inner layer and the material layer may be co-extensive about an entirety of the securing device. The inner layer and the material layer may be stretchable. The cavity of each of the projections define a pocket having an opening. The pockets may include a securing element to retain the pocket in a closed configuration.

The securing device may be configured such that an upper circumference about an upper edge of the securing device is less than or equal to a lower circumference about a lower edge of the securing device. The securing device may include a front portion configured to be positioned over the wearer's forehead, and the front portion may include an edge defining a curve when the securing device is laid flat. The front portion may have an upper edge defining a first curve and a lower edge defining a second curve, when the securing device is laid flat. A transverse, linear length of the first curve of the upper edge may be less than or equal to a second transverse, linear length of the second curve of the lower edge.

The securing device may include a lower edge that has a front portion defined by a convex downward curve, two projections defined by convex downward curves, and a back portion defined by a concave downward curve. A height of the securing device at the back portion may be less than or equal to a front height of the securing device at the front portion, both of which are less than a projection thickness at the projections.

The securing device may include an upper edge of the body having a front portion defined by a concave upward curve when laid in a flat position.

The securing device may include a front portion the includes a downward taper to approximate the curved sections. For ease of manufacturing, the front portion may be linearly or near linearly cut at an angle relative to the remaining or back portion of the body portion, such as relative to the upper edge. In this way the front portion is generally ‘v’ shaped, but is a shallow cut that it can approximate a concave upward curved as described herein. As used herein, an exemplary embodiment includes a front portion that includes a downward taper. The downward taper is understood to encompass a plurality of linear cuts to generally create a shallow ‘v’ configuration, a curved cut to create a shallow ‘u’ configuration, and combinations thereof to approximate the curved effect to securely fit relative to the user's head.

The securing device according to embodiments described herein may include components and/or characteristics that facilitate the desired benefits. For example, the securing device may be configured as a headband including an inner layer that facilitates temperature (either heat or cold) transfer to the user. The inner layer may also have a material quality, such as being breathable or soft. The headband may include an outer layer that facilitates compression and conforming of the headband and the inserted pack around the user's ear. The outer layer may also include other material qualities, such as insulation to reduce the dissipation of heat or cold to the environment and direct the thermal effects toward the user and away from the environment.

In an exemplary embodiment, a head band includes features to compress up to two hot or cold packs within two designated pockets specifically positioned over the user's ears. The material of the head band is made from a flexible material such as nylon or spandex for maximum comfort while being worn directly on top of the user's head. The top of the headband can have a smaller circumference (approximately 18″) versus the bottom (approximately 19″) to allow the head band to fit more comfortably to the user.

Similar to the front of the head band, the back of the head band is noticeable narrower than the pockets that directly cover the user's ears. The back of the head band can be a fixed hem or it can be an adjustable system that uses predetermined configurations made from plastic snaps, buttons, magnets, hook and loop fasteners, belts, sliders, or other materials that allow the user to adjust the circumference of the main head band body to fit larger or smaller sized heads. In some embodiments, the inner material of the pockets continue to run along other parts of the head band that are not directly part of the pockets including the fore head and the back of the head band.

According to exemplary embodiments, each hot/cold packs may include an outer shell that encapsulates a liquid gel material inside. When warmed in a microwave or oven, the liquid gel can hold a higher temperature longer than water. Conversely, when the pack is put inside a freezer until solid, the liquid gel found within the hot/cold packs will retain its lower temperature longer than water. In each case, the properties of the liquid gel are desirable because it allows the user to benefit from the temperature gradient from each hot/cold pack for the suggested length of time of 12-18 minutes while being worn on top of the user's head. The gel within the hot/cold pack may also be configured to not fully solidify such that it can deform and conform to the wearer's head, even after being cooled in a freezer.

Different features and configurations of the securing device have been described herein. Any combination of features, configurations, sizes, shapes, and/or components may be used in any combinations. The specific combinations described herein are exemplary only. Some embodiments of the head band include the following features that may be used in any combination:

Adjustable backing system may be used to allow the user to adjust the circumference of the main body to be either larger or smaller. The adjustment system can result from a buttons, snaps, buckle, slider, clips, or other types of systems that could be positioned along the head band—either near the front or the back—that would allow the user to adjust the circumference of the head band for a more desirable fit.

Additional material, reinforcement or removeable inserts within the pockets to help better insulate the temperature element to allow the user to maintain a desirable core temperature longer. The additional material may be used in place of the temperature element in the case of providing insulation and/or noise reduction, for example. Any of the additional material, insulation, noise reduction, temperature element may be generally referred to herein as an insert.

Layering the inside of the pockets with a material that would allow it to fasten securely and directly to the inserted temperature element and/or additional material (the insert). A complimentary material could also be added to the insert to help pair the two bodies together. This would allow the user to quickly discover specific placement of each insert depending on the application and purpose.

A separate folded flap or opening (not designed to hold a hot/cold pack) either on the inside or the outside of the securing device that would allow the head band to be folded within itself for reducing its size during storage.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

As used herein, the terms “about,” “substantially,” or “approximately” for any numerical values, ranges, shapes, distances, relative relationships, etc. indicate a suitable dimensional tolerance that allows the part or collection of components to function for its intended purpose as described herein. Numerical ranges may also be provided herein. Unless otherwise indicated, each range is intended to include the endpoints, and any quantity within the provided range. Therefore, a range of 2-4, includes 2, 3, 4, and any subdivision between 2 and 4, such as 2.1, 2.01, and 2.001. The range also encompasses any combination of ranges, such that 2-4 includes 2-3 and 3-4.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components or portions thereof. Such terminology may include the words specifically mentioned above, derivatives thereof, and words of similar import. The descriptions of first, second, third, etc. are not intended to define or require a specific number of components or event require one to be separate from another. They are merely indicate to describe a component with specific features that may or may not be separate or different from another component with the same or different features.

Conditional language used herein, such as, among others, “can,” “could,” “might,” “may,” “e.g.,” and the like, unless specifically stated otherwise, or otherwise understood within the context as used, is generally intended to convey that certain embodiments include certain features, elements and/or states. However, such language also includes embodiments in which the feature, element or state is not present as well. Thus, such conditional language is not generally intended to imply that features, elements and/or states are in any way required for one or more embodiments or that one or more embodiments necessarily exclude components not described by another embodiment.

1. (canceled)

2. A securing device configured to be positioned on a head of a wearer comprising:
a body configured to circumscribe the head of the wearer having a front portion, two opposing side portions, and a rear portion;
a first projection having a first cavity with a first cavity opening positioned on a first side of the body and a second projection having a second cavity with a second cavity opening positioned on the second side of the body;
a securing element;
a temperature element having a positioning element extending from a surface of the temperature element configured to engage the securing element,
wherein the securing element secures the temperature element within at least one of the first cavity and the second cavity at a location away from a bottom edge of the first cavity or the second cavity.

3. The securing device of claim 2, wherein the temperature element is at least one of a heating element and a cooling element.

4. The securing device of claim 1, wherein the body has an inner layer comprising apertures to facilitate thermal transfer from the temperature element to the head of the user.

5. The securing device of claim 4, wherein the inner layer is co-extensive about an entirety of the securing device.

6. The securing device of claim 5, wherein the securing device is stretchable.

7. The securing device of claim 2, wherein the securing element retains the pocket in a closed configuration.

8. The securing device of claim 7, wherein an upper circumference about an upper edge of the securing device is less than or equal to a lower circumference about a lower edge of the securing device.

9. The securing device of claim 2, wherein a lower edge of the body comprises a front portion defined by a convex downward curve, two projections defined by convex downward curves, and a back portion defined by a concave downward curve.

10. The securing device of claim 10, wherein a back height of the securing device at the back portion is less than or equal to a front height of the securing device at the front portion, both of which are less than a projection thickness at the projections.

11. The securing device of claim 2, wherein an upper edge of the body comprises a front portion defined by a concave upward curve when laid in a flat position.

12. The securing device of claim 2, wherein the body includes a front portion that is tapered downward.
